(19) United States
(12) Patent Application Publication
(10) Pub. No.: US 2021/0023329 A1
(43) Pub. Date: Jan. 28, 2021

(54) DEVICE AND METHOD FOR TRAINING THE MUSCLES AND CONNECTIVE TISSUE IN THE ORAL CAVITY AND THROAT OF A PERSON, PARTICULARLY FOR LONG-TERM AVOIDANCE OF AIRWAY AND SLEEP DISORDERS AND THE CONSEQUENCES THEREOF

(71) Applicant: Asate AG, Jona (CH)

(72) Inventors: Alexandre SUAREZ, Baeretswil (CH); Aron SUAREZ, Schmerikon (CH)

(73) Assignee: Asate AG, Jona (CH)

(21) Appl. No.: 16/492,252

(22) PCT Filed: Mar. 9, 2018
(86) PCT No.: PCT/CH2018/000007

§ 371 Date: Sep. 9, 2019

(30) Foreign Application Priority Data
Mar. 13, 2017 (EP) 17160644.5

(51) Int. Cl. A61M 21/02; G10D 7/06; G10H 3/14

(52) U.S. Cl. CPC A61M 21/02; A61M 2021/0027; G10H 3/143; G10D 7/06

(57) ABSTRACT

A device for training the muscles and connective tissue in the oral cavity and throat of a person, particularly for long-term avoidance of airway and sleep disorders and the consequences thereof, includes a wind instrument and furthermore an apparatus for detecting a sound produced by a person by the wind instrument. The device further includes an apparatus for evaluating the detected sound and an apparatus for outputting a response defined in dependence on a result of the evaluation to the person playing the wind instrument.

The present invention relates to an apparatus and a method for training the muscles and connective tissue in the oral cavity and throat of a person, particularly for long-term avoidance of airway and sleep disorders and the consequences thereof.

The company Asate AG in Schmerikon, Switzerland has introduced the concept of training the muscles and connective tissue in the oral cavity and throat of a person mainly for the purpose of avoiding respiratory tract disorders, possibly with the aid of a wind instrument. As a result, it is no longer necessary to perform operative interventions on oral cavity and throat muscles. Also there is no longer any need for aids used nightly which serve to keep the airways open during sleep.

In the field of teaching materials for musical instruments, it is known to use a personal computer. Thus, for example, an electric guitar can be coupled to such a personal computer, it plays accompanying music and notes to be played are displayed to the learner.

It is the object of the present invention to provide an improved apparatus and an improved method for training the muscles and the connective tissue in the oral cavity and throat of a person.

This object is solved by an apparatus having the features of patent claim 1 and a method having the features of patent claim 14.

According to the invention in addition to a wind instrument, a device for detecting a sound produced by a person by means of the wind instrument is provided, a device for evaluating the detected sound is provided and finally there is a device for outputting a response defined as a function of a result of the evaluation to the person playing the wind instrument. With the apparatus according to the invention, an at least partial monitoring of a training is made possible. The training person need not himself know whether he has hit the notes or blown sufficiently firmly but experiences this by means of the device for evaluation and a response is given to the person. Thus, a training program can be completed much more precisely. In turn, the more precisely a devised training program is now followed, the better the success that can be achieved when training the muscles and the connective tissue, in particular airway and sleep disturbances can be avoided in the long term with a higher probability.

In a preferred embodiment of the invention, the device for evaluating comprises a data storage device in which at least one file with information is stored which gives target specifications provided for the playing of the wind instrument. In this way, the device for evaluating can evaluate the detected sound according to specific criteria, namely according to the target specifications.

The target specification can also be output to the person playing the wind instrument. For the latter purpose in a further preferred embodiment it is provided that the device for evaluating is coupled to the device for outputting and the device for outputting is designed to output a message to the person playing the wind instrument by means of which a target specification is given to the person. The person playing the wind instrument therefore need not know the target specification himself but receives relevant information during training.

Furthermore, with regard to the embodiment with the data storage device it is preferred if the information in the at least one file relating to at least a portion of the target specifications in each case also specifies which condition must be satisfied by the data relayed from the device for detecting the sound to the device for evaluating so that this target specification is satisfied. The device for evaluating is then designed to check that this condition is satisfied. This preferred embodiment is based on the fact that no target specification can always and perfectly be implemented but that a certain tolerance can be given. This can, for example relate to a pitch level of the sound produced by the wind instrument and/or a loudness: for the pitch level or loudness an interval is given in each case which predefines a range of validity. If the pitch level or loudness falls in the interval, the target specification is deemed to be satisfied.

In a preferred embodiment of the apparatus according to the invention, the device for detecting comprises a microphone arranged in or on the wind instrument and/or a piezoelectric sensor arranged in or on the wind instrument. By integrating the device for evaluating into the wind instrument, a particularly easy to handle wind instrument is provided and a coupling with a separate device for detecting is not necessary.

In the said embodiment, it is preferably provided that the device for detecting comprises a transmitting device coupled to the microphone or the piezoelectric sensor and arranged in or on the wind instrument for transmitting data recorded with the aid of the microphone or the piezoelectric sensor to the device for evaluating. In this way, provision is also made for the output of the corresponding data in or on the wind instrument, i.e. no additional device is required.

In the said embodiment it is further preferred if the device for evaluating is provided outside the wind instrument so that it is possible to use such a device which need not be produced separately for the wind instrument. This is, for example, a smartphone, a tablet computer or a personal computer. Alternatively to this embodiment it would also be possible however to also provide the device for evaluating in or on the wind instrument. Such an embodiment would have the advantage that one would be dealing with a very compact device.

In the device for evaluating provided outside the wind instrument, the data transfer takes place in one variant via cable. In another variant, the transmitting device is designed to transmit the data recorded by means of the microphone or the piezoelectric sensor wirelessly to the device for evaluating. In this case, for example, conventional Bluetooth® devices can be used.

In order that the wind instrument can also be played without having a power cable plugged in continuously, a storage device for electrical energy is preferably provided in the wind instrument in order to firstly operate the microphone or the piezoelectric sensor and/or secondly operate the optionally provided transmitting device.

In the apparatus according to the invention, a further preferred embodiment includes the fact that the device for evaluating the recorded sound and a device (42, 52) for outputting a response defined as a function of the result of the evaluation to the person playing the wind instrument is provided by a mobile data processing device (such as possibly a smartphone or a tablet computer), on which a corresponding program, (an app, an “application”) is stored. In this way, the display pertaining to such a data processing device (touchscreen or the like) can be used to give a response to the player of the wind instrument.

In a preferred embodiment of the apparatus according to the invention, the wind instrument has a plurality of bends consecutively in succession. It can prove expedient here if there are between five and twelve bends, for example between seven and ten bends, e.g. nine.

In the apparatus according to the invention, it is further provided in one variant that the wind instrument consists of wood at least in sections and preferably in the sound-producing part (possibly pipe and resonating body) completely consists of wood. In another variant, the wind instrument consists of plastic at least in sections preferably in the sound-producing part (possibly pipe and resonating body) completely consists of plastic. Both materials can also be combined with one another (e.g. pipe made of plastic and resonating body made of wood or mouthpiece on the pipe made of wood and all others made of plastic. In this way the wind instrument can be produced cost-effectively and can nevertheless have a good sound quality.

The method according to the invention for training the muscles and the connective tissue in the oral cavity and throat of a person, in particular for long-term avoidance of airway and sleep disorders and the consequences thereof, comprising the steps:
providing a wind instrument,
providing a sound recording device, a data processing device as evaluation device, an output device and a data storage device in which at least one file with information is stored which gives target specifications provided for the playing of the wind instrument and wherein the information in the at least one file relating to at least a part of the target specifications specifies in each case which condition must be satisfied by data relayed from the sound recording device to the evaluation device so that this target specification is satisfied,
outputting a target specification by the output device for perception by the person,
receiving sound signals by the sound recording device and relaying corresponding data to the evaluation device,
investigating the data to determine whether the condition specified for the output target specification is satisfied by the evaluation device,
outputting a response by the output device for perception by the person as a function of the result of the investigation.

The method according to the invention includes the fact that the person knows which sounds (and how) they are to be played through the output of the target specification by means of the output device. A response (“feedback”) is then made so that the person can optimize the playing and therefore also the training in order to train their muscles and their connective tissue in the oral cavity and throat so that airway and sleep disorders can be avoided in the long term.

In a preferred embodiment of the method according to the invention, the step of outputting a target specification is preceded by:
a) receiving an input of the person for selection of a training program and/or
b) recording the subject and/or the time for at least one earlier target specification and/or
c) recording the subject and/or the time for at least one earlier response,
   wherein the output target specification is output depending on at least one of the input, the at least one recorded subject of an earlier target specification, the at least one recorded time of an earlier target specification, the at least one recorded subject of an earlier response and the at least one recorded time of an earlier response. Thus, the person can seek out a specific training program or however additionally or alternatively the training program is dependent on the previously covered training and/or the previous training success. Alternatively to this, the target specification can be dependent on an absolute time, possibly defined by the date. Then a continuous training program is simply provided regardless of whether the person is actually training (and how frequently) or not. If the person has paused for three days, for example, the training demands in between have possibly increased which then makes the training after the pause much more demanding.

In a further preferred embodiment of the method according to the invention, the condition specified for a target specification varies as a function of at least one of the subject and time of an earlier target specification and subject and time of an earlier response. Thus, the training can take place more flexibly. If the person playing the wind instrument does not reach the pitch level and/or loudness quite as desired, the training program can set out somewhat coarser criteria. If the person reaches the pitch level and loudness particularly well, conversely the requirement can also gradually be increased. Alternatively to this, a continuous training program is specified in advance. However, the results of measurements on the person (possibly imaging in the oral cavity and throat) and/or with the involvement of the person (“trial blowing”) can be included in the specification.

Further details of the invention and in particular exemplary embodiments of the apparatus according to the invention and the method according to the invention are explained hereinafter with reference to the appended drawings, In the figures:

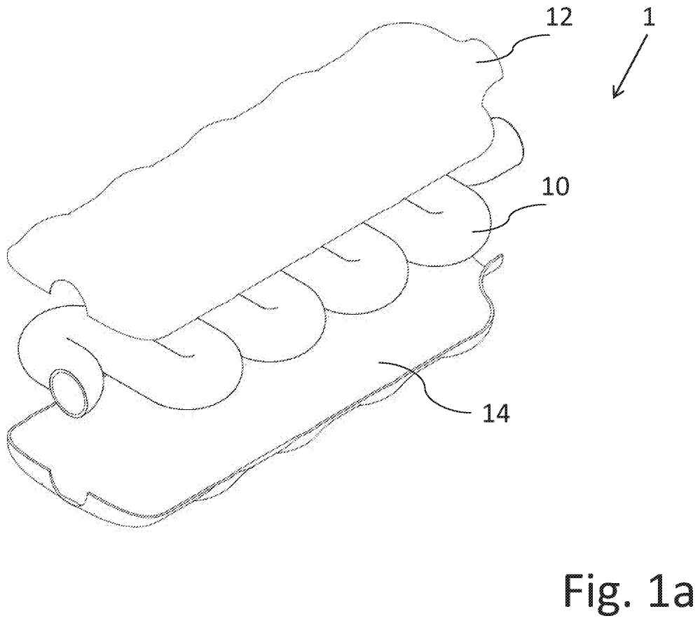

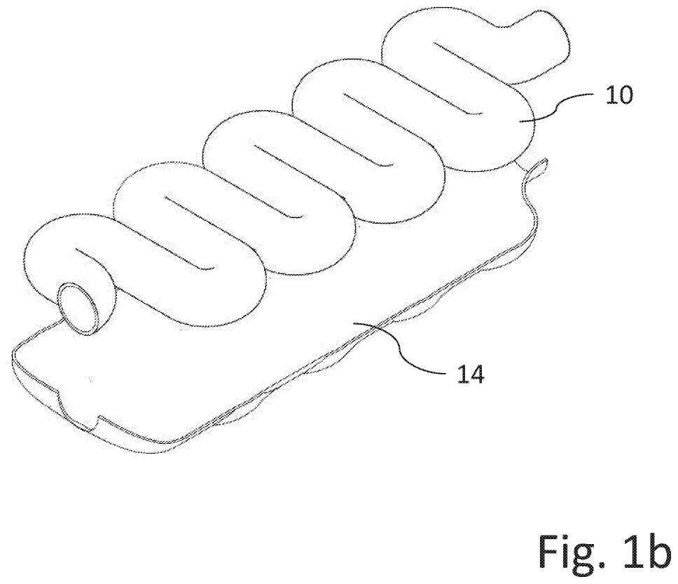

FIG. 1a shows an exploded view of a wind instrument such as can be seen in the apparatus according to the invention,

FIG. 1b shows an exploded view of the wind instrument from FIG. 1a without its top cover,

FIG. 1c shows the blowing body of the wind instrument from FIG. 1b in perspective view,

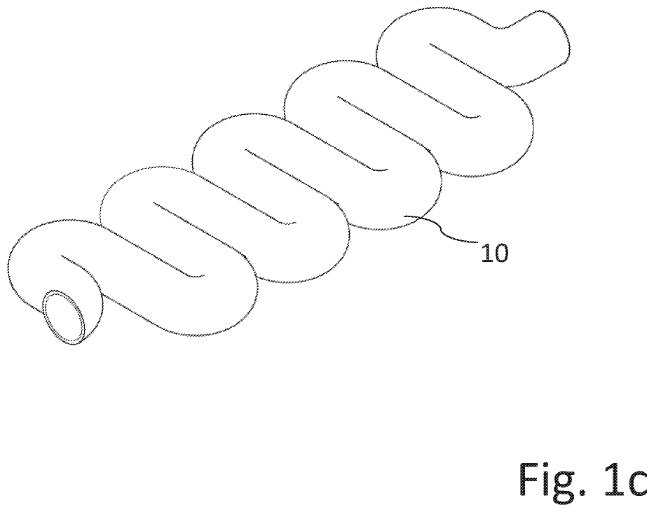

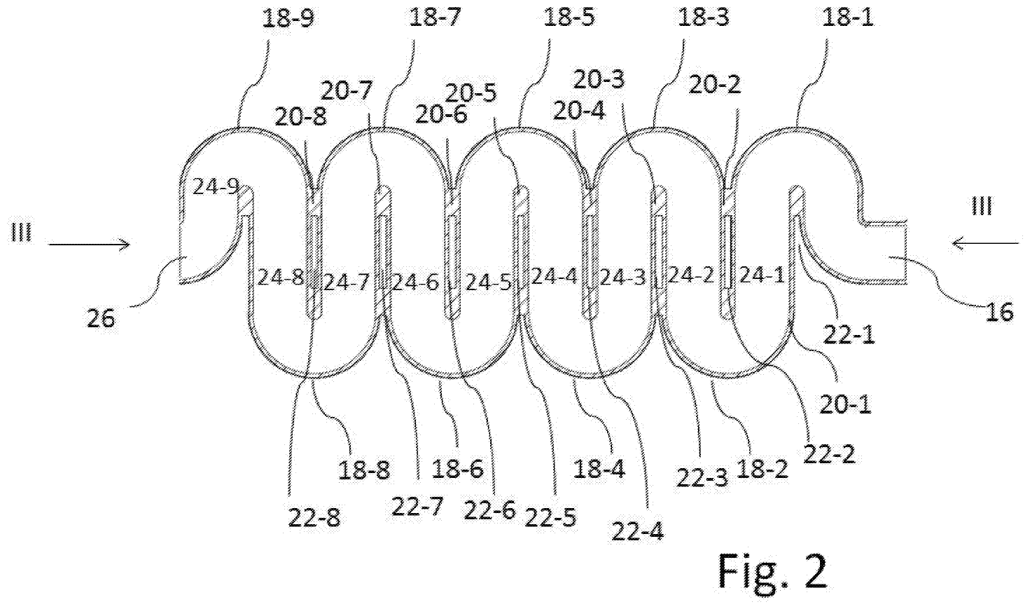

FIG. 2 shows a section through the blowing body from FIG. 1c,

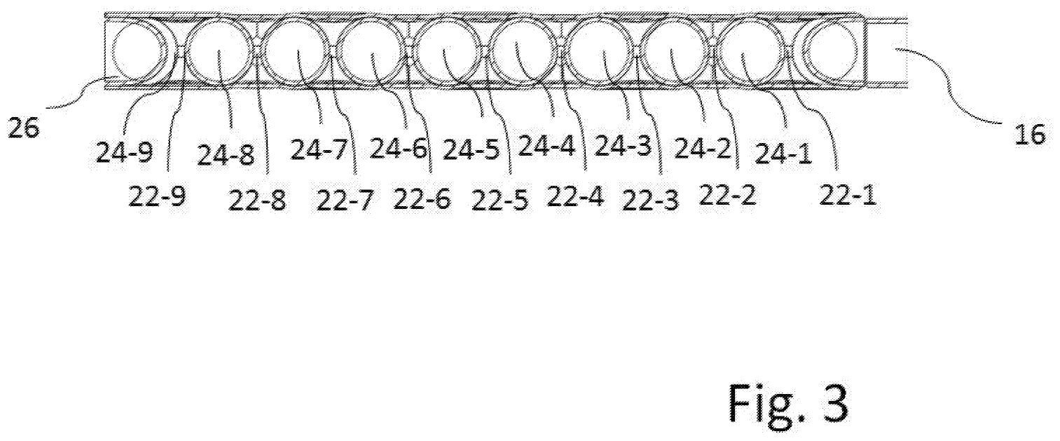

FIG. 3 shows a lateral section through the wind instrument shown in FIG. 1a in the assembled state,

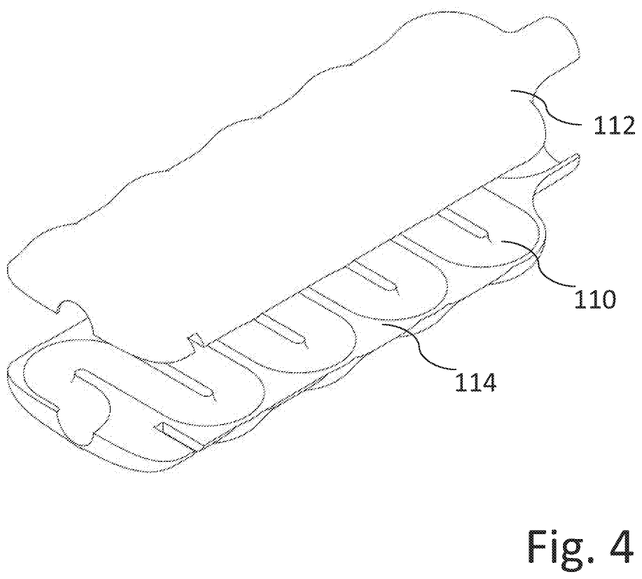

FIG. 4 shows an alternative embodiment of cover and base of the wind instrument which is shown in FIG. 1a,

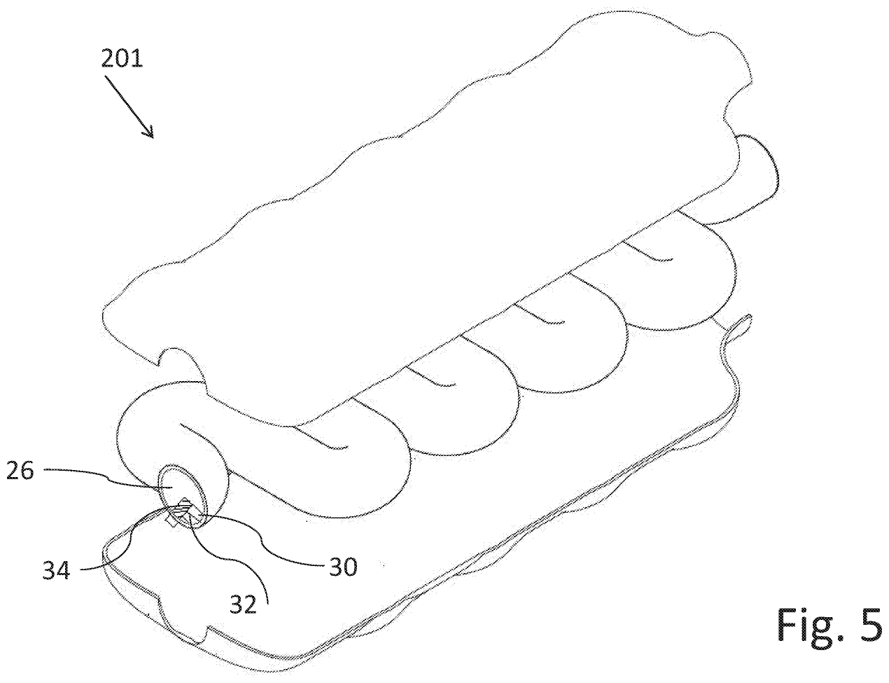

FIG. 5 shows the wind instrument from FIG. 1a with additional devices such as are used in a first embodiment of the apparatus according to the invention,

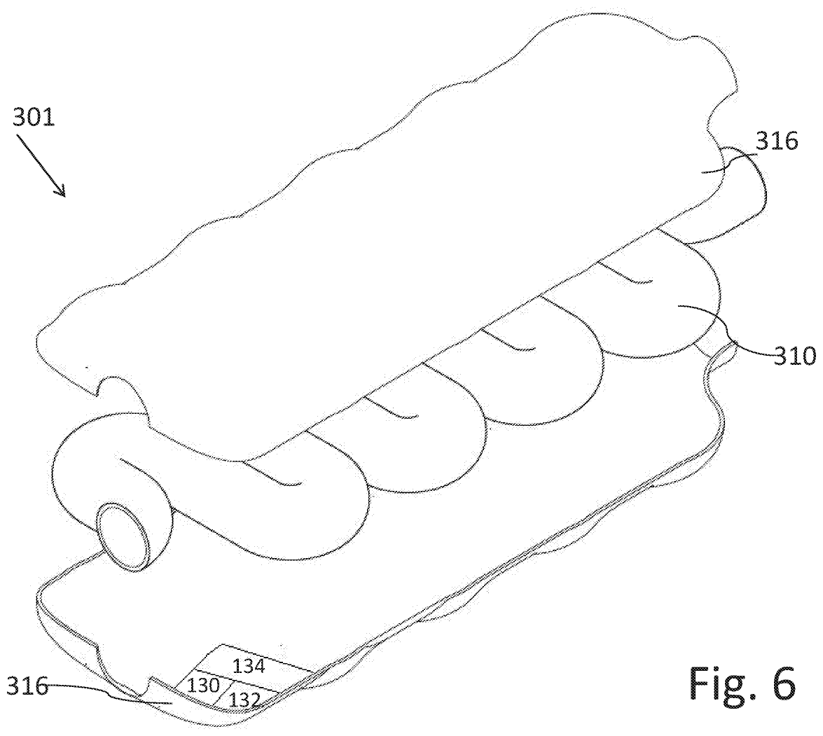

FIG. 6 shows the wind instrument from FIG. 1a with additional devices such as are used in a second embodiment of the apparatus according to the invention,

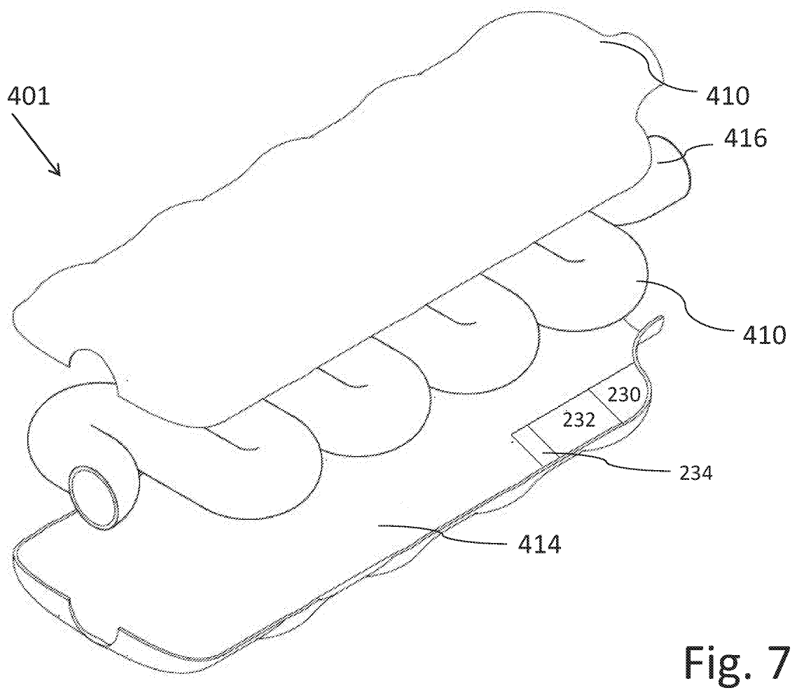

FIG. 7 shows the wind instrument from FIG. 1a with additional devices such as are used in a third embodiment of the apparatus according to the invention,

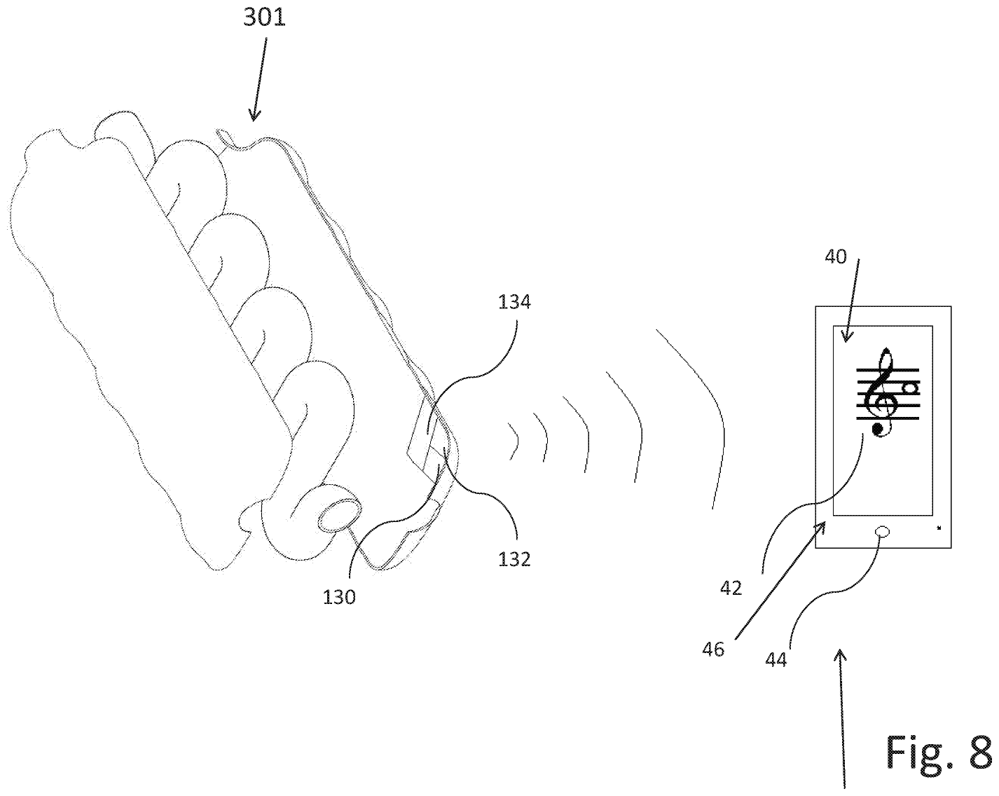

FIG. 8 shows the complete apparatus according to one embodiment of the invention using a smartphone and

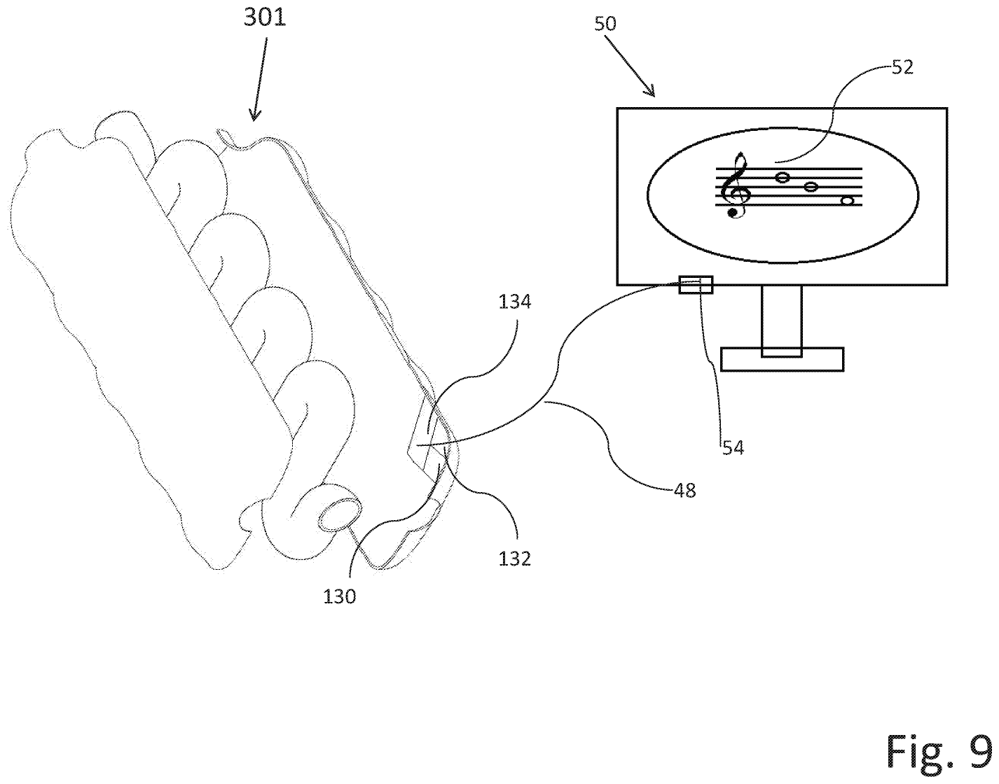

FIG. 9 shows the complete apparatus according to a further embodiment of the invention using a personal computer,

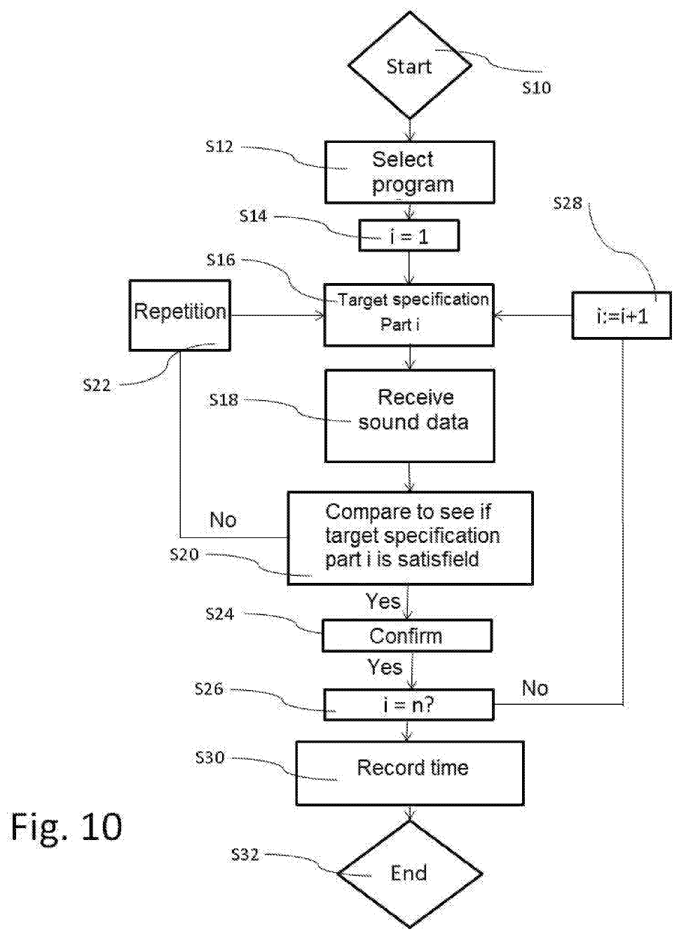

FIG. 10 shows a flow diagram to illustrate an embodiment of the method according to the invention.

The wind instrument shown in exploded view in FIG. 1a and designated overall by 1 comprises the actual blowing body 10, an top cover 12 and a base 14. FIGS. 1b and 1c each show only parts of the wind instrument to make it more easily viewed.

The blowing body 10 is shown in section in FIG. 2. It comprises an inlet connector 16 which is followed in the direction of ingress of the air during blowing by a first bend 18-1, followed by a second bend 18-2, and further bends 18-3, 18-4, 18-5, 18-6, 18-7, 18-8 and 18-9. Located between the two ends 18-1 and 18-3 is a rectilinear wall section 20-1, between the bends 18-2 ad 18-3 a rectilinear wall section 20-2 and rectilinear wall sections 20-3, 20-4, 20-5, 20-6 and 20-7 respectively stand between pairs of bends. An open space 22-1 is located between the mouthpiece 16 and the rectilinear wall section 20-1. Cavities 22-2, 22-3, 22-4, 22-5, 22-6, 22-7, 22-8 are located in the region of the rectilinear wall sections 20-2, 20-3, 20-4, etc. The cavities can but need not be configured so that they act as resonant chambers and contribute to the amplification of the sound. Located between the rectilinear wall sections 20-1 and 20-2, is a pipe section 24-1, between the rectilinear wall sections 20-2 and 20-3, the pipe section 24-2, etc. The last pipe section 24-8 goes over into a pipe section 24-9 which finally opens into the outlet connector 26.

FIG. 3 shows in the section III-III from FIG. 2 the respective pipe sections 24-1, 24-2, 24-3 etc. with the chambers 22-3, 22-4 etc. located in between. The pipe sections are of circular cross-section and have a diameter of between 1 and 5 cm, for example, of between 2.2 and 2.8 cm.

In the alternative embodiment of the wind instrument according to FIG. 4, only two parts are provided, namely the top cover 112 and the base 114, wherein the wind instrument 101 is provided by cavities 110 in the base which are supplemented by corresponding (not shown in the figure) cavities in the top cover to form the passage space for air in the wind instrument 101.

The apparatus according to the invention includes a sound receiving device by means of which the sound is recorded. Both the embodiment 1 according to FIG. 1a and the embodiment 101 according to FIG. 4 can cooperate with an external sound receiving device. Hereinafter however those embodiments are described as preferred embodiments in which a sound-receiving device is part of the wind instrument. This is explained by reference to modifications of the wind instrument 101 from FIG. 1a, wherein the embodiments described here can also be used adapted accordingly in the alternative 101 according to FIG. 4.

In a first variant of the wind instrument 101 with built-in sound recording device as shown in FIG. 5 and designated there by 201, in the region of the outlet connector 26 for air a microphone 30 is provided to which a transmitting device 32 is assigned, wherein microphone 30 and transmitting device 32 are supplied with energy via an energy storage device 34 (for example, in the form of a lithium ion rechargeable battery). The energy storage device 34 can either be exchangeable or can be coupled to an external power source such as a plug socket by connection of a charging cable not shown to be charged.

In the variant according to FIG. 6, which is designated overall by 301, a microphone 130 is provided in a cavity in the base 314 outside the blowing body 310 and is coupled to a wall 316 of the base in the vicinity of the outlet connector 326 to record sound produced therein. The transmitting device 132 and an energy storage device 134 are also provided in this region.

In a further variant shown in FIG. 7 and designated there by 401, a microphone 230, a transmitting device 232 and an energy storage device 234 are also provided in a cavity of the base 414, outside the blowing body 410, wherein here the microphone 230 is located in the vicinity of the inlet connector 416.

The implementation of the invention is explained by reference to FIGS. 8 and 9, which is shown linked to the embodiment 301 of the wind instrument according to FIG. 6 but can be implemented equally well in the other embodiments according to FIG. 5 or 7. When blowing the wind instrument, the microphone 130 records the sound thus produced and the transmitting device 132 transmits the recorded sound data in a wireless manner, for example, using a Bluetooth® standard to a corresponding receiving device 46 of a smartphone 40. The smartphone comprises a display 42. A note is shown as an example on the display 42 to symbolize that a specification is provided to the person playing the wind instrument (i.e. the person training his muscles and connective tissue in the oral cavity and throat) as to which sound he has to play. Inputs can additionally be made via an input device 44 such as a switch, touchscreen or the like on the smartphone 40.

In the alternative embodiment according to FIG. 9, the transmitting device 132 is coupled via a cable 48 to a personal computer 50 which has a screen 52 and an interface 54 for the cable.

The method according to the invention begins in step S10 (“start”) with the activation of the electronic part, for example, switching on the wind instrument with a suitable switch not shown in the figures and switching on the smartphone 40 or the personal computer 50. In step S12 the selection of a program, namely a training program is made by the trainee. Alternatively he simply activates a single training program, possibly be switching in an app. This is a training program which provides specifications to the user as to how he should play the wind instrument in order to train the muscles and the connective tissue in the oral cavity and throat optimally so that airway and sleep disturbances are avoided in the long term. Here it is a question of running through a specific training program as accurately as possible. Each program comprises a number n of target specifications. It begins with the first (i:=1 is set in step S14). In step S16 the target specification, part i, is output via the display 42 of the smartphone or the screen 52 of the personal computer. The trainee now actuates the wind instrument by blowing into the inlet connector 416 and with the aid of the microphone 132 which is part of the wind instrument 101 in the exemplary case of the figures, the sound data are received in step S18. An evaluation device (a data processing processor or the like in the smartphone 40 or in the personal computer 50) compares whether the target specification is satisfied according to a predetermined criterion. For this purpose in a data storage device not shown a region, can be specified for each target specification which defines whether the target specification is satisfied. For example, the target specification can include that a specific pitch level should be played, then adjacent pitch levels can also be deemed to be acceptable. Thus, in a note system the sound to be played can be displayed (possibly in green) and the sound actually played can be additionally displayed (possibly in red). If the sound played is too low, for example, the sound played is indicated with a red colour in the note system. If the player raises the sound, the note moves upwards in the diagram and turns green as soon as it falls within the defined sound interval.

Furthermore for example, a specific loudness can be predefined, which can be achieved, then related loudnesses can also be achieved. The implementation of a specific loudness can also be accomplished by means of continuous feedback: typically a bar diagram will be selected here in which the length of the bar corresponds to a loudness; the longer the bar, the louder is the loudness. A green dash is displayed for the loudness to be achieved. The bar remains red until the interval in which the loudness should lie is reached and then the bar is coloured green. Other types of representation are naturally also possible. The satisfaction of the target specification can also be defined by a combination of pitch level and loudness which for example could be given as 2-tuple. (loudnesses can be given which are accepted for one specific pitch level but not for another pitch level).

Alternatively only one signal can be simply output in a simplifying manner which displays to the trainee that he should in fact blow into the instrument. The signal can be audible (possibly a horn sound or peep sound) or visual (lights or flashing of a lamp and the like). Subsequently in each passage respectively one sound or a sound sequence can be pre-played to the trainee which he should reproduce by blowing.

In step S20 it is checked whether the target specification, part i is satisfied. If this is not the case, a repetition is made according to step S22 and a return to step S10. Here the number of repetitions can possibly be counted to allow an exit scenario which however is not shown in FIG. 10. If on the other hand the target specification is satisfied in step S20, in step S24 an output is made to the trainee to indicate the success. It is further checked whether this is the last target specification (i=n). This check is made in step S26. If the event is negative, the variable i is increased by 1, step S28 includes i:=(i+1) and a return is made to step S16 which displays the next target specification, sound data are received again and a check is again made to see it the target specification is satisfied. Finally if i=n is achieved, the time of reaching this (date and time) is recorded in step S30; optionally additional information can be recorded such as the number of repetitions according to step S2 or the total duration required by the trainee to run through all the target specifications. With this information after the next start the user can be given a suitable specification for the selection of programs. Finally all the electrical equipment is switched off and the method ends in step S32.

The method according to the invention can also manage without any previous specification of the number of target specifications, i.e. n is set so high that it is never reached. The trainee can then independently end the training.

Overall the method described by means of FIG. 10 is only exemplary and when implemented, some steps and aspects can be omitted or others added.

1. An apparatus for training the muscles and the connective tissue in the oral cavity and throat of a person, in particular for long-term avoidance of airway and sleep disorders and the consequences thereof, comprising a wind instrument (1, 101, 201, 301, 401) and
comprising a device (30, 130, 230) for detecting a sound produced by a person by means of the wind instrument (1, 101, 201, 301, 401), a device (40, 50) for evaluating the detected sound and a device (42, 52) for outputting a response defined as a function of a result of the evaluation to the person playing the wind instrument (1, 101, 201, 301, 401).

2. The apparatus according to claim 1, in which the device for evaluating comprises a data storage device in which at least one file with information is stored which gives target specifications (S16) provided for the playing of the wind instrument (1, 101, 201, 301, 401).

3. The apparatus according to claim 2, in which the device (40, 50) for evaluating is coupled to the device (42, 52) for outputting and the device (42, 52) for outputting is designed to output a message to the person playing the wind instrument (1, 101, 201, 301, 401) by means of which a target specification is given to the person.

4. The apparatus according to claims 2 and 3, in which the information in the at least one file relating to at least a portion of the target specifications in each case also specifies which condition must be satisfied by the data relayed from the device (30, 130, 230) for detecting the sound to the device (40, 50) for evaluating so that this target specification is satisfied and in which the device (40, 50) for evaluating is designed to check that the condition is satisfied.

5. The apparatus according to claim 1 in which the device for detecting comprises a microphone (30, 130, 230) arranged in or on the wind instrument and/or a piezoelectric sensor arranged in or on the wind instrument.

6. The apparatus according to claim 5, in which the device for detecting comprises a transmitting device (32, 132, 232) coupled to the microphone (30, 130, 230) or the piezoelectric sensor and arranged in or on the wind instrument for transmitting data recorded with the aid of the microphone or the piezoelectric sensor to the device (40, 50) for evaluating.

7. The apparatus according to claim 6, in which the device (40, 50) for evaluating is provided outside the wind instrument.

8. The apparatus according to claim 7, in which the transmitting device is designed to transmit the data recorded by means of the microphone or the piezoelectric sensor wirelessly to the device (40, 50) for evaluating.

9. The apparatus according to claim 5, in which the wind instrument (1, 101, 201, 301, 401) has a storage device (34, 134, 234) for electrical energy in order to firstly operate the microphone (30, 130, 230) or the piezoelectric sensor and/or secondly operate the transmitting device (32, 132, 232).

10. The apparatus according to claim 1 in which the device for evaluating the recorded sound and a device (42, 52) for outputting a response defined as a function of the result of the evaluation to the person playing the wind instrument is provided by a mobile data processing device (40), in particular a smartphone (40) or a tablet computer, on which a corresponding program, in particular an app is stored.

11. The apparatus according to claim 1 in which the wind instrument (1, 101, 201, 301, 401) has a plurality of bends (18-1, 18-2, . . . , 18-9) consecutively in succession, preferably between five and twelve bends.

12. The apparatus according to claim 1 in which the wind instrument (1, 101, 201, 301, 401) comprises wood at least in sections or plastic at least in sections.

13. A method for training the muscles and the connective tissue in the oral cavity and throat of a person, in particular for long-term avoidance of airway and sleep disorders and the consequences thereof, comprising the steps:
providing a wind instrument (1, 101, 201, 301, 401),
providing a sound recording device (30, 130, 230), a data processing device (40, 50) as evaluation device, an output device (42, 52) and a data storage device in which at least one file with information is stored which gives target specifications provided for the playing of the wind instrument (1, 101, 201, 301, 401) and wherein the information in the at least one file relating to at least a part of the target specifications specifies in each case which condition must be satisfied by data relayed from the sound recording device (30, 130, 230) to the evaluation device so that this target specification is satisfied,
outputting a target specification by the output device (42, 52) for perception by the person,
receiving sound signals by the sound recording device (30, 130, 230) and relaying corresponding data to the evaluation device (40, 50),
investigating the data to determine whether the condition specified for the output target specification is satisfied by the evaluation device (40, 50), and
outputting a response by the output device (42, 52) for perception by the person as a function of the result of the investigation.

14. The method according to claim 13, in which the step of outputting a target specification is preceded by:
a) receiving an input of the person for selection of a training program and/or
b) recording the subject and/or the time for at least one earlier target specification and/or
c) recording the subject and/or the time for at least one earlier response,
wherein the output target specification is output depending on at least one of the input, the at least one recorded subject of an earlier target specification, the at least one recorded time of an earlier target specification, the at least one recorded subject of an earlier response and the at least one recorded time of an earlier response.

15. The method according to claim 13, in which the condition specified for a target specification varies as a function of at least one of the subject and time of an earlier target specification and subject and time of an earlier response.
